(19) United States
(12) Patent Application Publication
(10) Pub. No.: US 2024/0390256 A1
(43) Pub. Date: Nov. 28, 2024

(54) A DEODORANT COMPOSITION

(71) Applicant: Conopco, Inc., d/b/a UNILEVER, Englewood Cliffs, NJ (US)

(72) Inventors: Anindya DASGUPTA, Bangalore (IN); Janhavi Sanjay RAUT, Bangalore (IN); Maya Treesa SAJI, Bangalore (IN)

(73) Assignee: Conopco, Inc., d/b/a UNILEVER, Englewood Cliffs, NJ (US)

(21) Appl. No.: 18/685,997

(22) PCT Filed: Sep. 2, 2022
(86) PCT No.: PCT/EP2022/074462

§ 371 Date: Feb. 23, 2024

(30) Foreign Application Priority Data
Sep. 6, 2021 (EP) 21195018.3

(51) Int. Cl. A61K 8/73; A61K 8/67; A61Q 15/00

(52) U.S. Cl. CPC A61K 8/732; A61K 8/675; A61Q 15/00

(57) ABSTRACT

The present invention relates to an antimicrobial composition, more particularly to a method and a composition to prevent or mitigate bodily malodour, especially present in human axilla. This is achieved by a composition that comprises a vitamin B3 compound or derivative thereof along with pectin.

### FIELD OF THE INVENTION

The present invention relates to an antimicrobial composition, more particularly to a method and a composition to prevent or mitigate bodily malodour, especially present in human axilla.

### BACKGROUND OF THE INVENTION

In humans, the skin of underarm provides a unique niche for bacteria. Through the secretions of various glands that open onto skin, the environment is nutrient rich and hosts a unique microbial community. In humans, the link between axillary apocrine gland secretions, underarm bacteria and body odour have been long established. Culture-based studies on this relationship found the axillary microbiota to be dominated by Staphylococcus, Corynebacterium and Propionibacterium species. More recent culture-independent studies have confirmed the presence of these genera and additionally, indicated the presence of Gram-positive anaerobic cocci (GPAC) belonging to Anaerococcus and Peptoniphilus genera. The primary reason for these studies was to identify target bacterial species responsible for axillary malodour and thereby design strategies to control it for providing the axillary malodour reduction benefit.

Human body odour contains several chemicals, but the most pungent and recognisable are thioalcohols. These molecules are created through a series of chemical reactions that start with an odourless precursor, a compound produced in glands located in the armpits. Popular theory has it that a type of bacteria called Staphylococcus hominis (S. hominis) takes in these molecules and transforms them into thioalcohols which give the body the malodour.

Body malodour has been tackled in many ways. One approach is to use perfumes to mask the malodour, but this approach has benefit only for a limited time. Anti-perspirant compositions are also available to minimize the formation of sweat on the skin. The present inventors in seeking to solve the problem of malodour were looking for a different approach to controlling the bacterial population in the underarm region. It is possible to completely eradicate or minimize all the bacteria on the skin through the use of agents like alcohol.

U.S. Pat. No. 3,369,551 (Robert G. Carroll, 1968) discloses a tobacco substitute base material which is an aqueous mixture of vitamin B compound or derivative thereof, and pectin.

CA1227430 (Colgate Palmolive Company) discloses a deodorant body cleaning composition comprising a detergent and a carbohydrate selected from glucose, mannose, and oligomers thereof.

JP2010088754 (Nicca Chemical Co. Ltd.) discloses a deodorizer composition for removing tobacco malodour and where the composition comprises pectin, L-ascorbic acid and an ultraviolet absorber.

AU2017294932 (Unilever PLC, 2018) discloses a deodorant composition having 0.5 wt. % to 10 wt. % niacinamide.

US 2020/316050 A1 (Unilever) discloses a composition having antimicrobial benefits and where the composition includes vitamin B3 compound.

WO 2019/086327 (Unilever) discloses a deodorant having niacinamide.

However, the present inventors wish to use more natural agents which are less harsh on skin. The present inventors thus started working towards providing “natural” solutions to solving the problem of malodour especially using the complex interaction of the skin microbiome with the various products that are known and used on skin for different benefits.

Pectin is used in personal care composition for various benefits like thickening and stabilizing semi solid compositions like gels, creams, and lotions. Biochemical benefits of pectin like providing barrier functions on skin and providing protective effect against water-loss have also been reported. To the knowledge of the present inventors, it is not known or reported that pectin has any effect on microbiome balancing benefit by way of targeted germ kill. A vitamin B3 compound, specifically niacinamide is known for various benefits like depigmentation, moisturization, generation of AMPs (anti-microbial peptides) thereby improving immunity, anti-inflammation, among a host of others. To the knowledge of the present inventors, vitamin B3 or any of its derivatives are not known for targeted inhibition of odour causing bacteria especially to the exclusion of skin commensal bacteria. It is in the backdrop of such information that the present inventors found the findings of the present invention to be highly surprising that two materials generally unknown for a specific benefit interacted to synergistically deliver that benefit.

It is thus an object of the present invention to provide for a composition that is effective on reducing malodour on skin while ensuring minimal impact on skin commensal bacteria.

### SUMMARY OF THE INVENTION

During the course of their work on understanding the various biochemical and microbiological interactions occurring on the skin microbiota, and through extensive experimentation, the present inventors have surprisingly found that a combination of pectin and a vitamin B3 compound synergistically interacted to give the desired benefit.

The combination not only provided the desired malodour reduction but also minimized negative impact on the rest the skin microbiome. Without wishing to be bound by theory, the inventors believe that this occurs as the inventive combination inhibits growth of the odour causing organism S. hominis while having minimal impact on skin commensal bacteria like S. epidermidis.

The first aspect of the present invention relates to a deodorant composition for reducing malodour on skin, the composition comprising
(i) pectin;
(ii) a vitamin B3 compound selected from the group consisting of niacinamide, homologues of niacinamide or mixtures thereof; and,
(iii) a cosmetically acceptable base.

Another aspect of the present invention relates to a method of inhibiting the growth of odour causing bacteria like S. hominis while not inhibiting the growth of skin commensal bacteria like S. epidermidis comprising the step of applying a composition of the first aspect on to a desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated.

Except in the operating and comparative examples, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated.

The composition as per this invention is intended for use as a deodorant composition, so preferably it is in the form of a leave-on format for delivering selective malodour benefit to topical areas e.g. skin and/or hair of mammals, especially humans. Leave-on composition means that the product is applied to the body without washing off with water at the time of application so that is left on the body surface for a substantial period of time of e.g. a few or several hours at least. It is especially effective for reducing axillary malodour. Such a composition includes any product applied to a human body for also improving appearance, cleansing, or general aesthetics. The composition of the present invention may be delivered with a topically acceptable base which could be an anhydrous base, liquid, lotion, cream, foam, scrub, gel, emulsion, or a propellant. “Skin” as used herein is meant to include skin on any part of the body (e.g., neck, chest, back, arms, underarms, hands, legs, buttocks, and scalp) especially the underarm. It is especially useful for reduction of malodour from the underarm (or axilla) or any other part of the body where malodour is generated.

The composition of the present invention comprises a vitamin B3 compound selected from niacinamide or homologues of niacinamide or mixtures thereof. The vitamin B3 compound selected from niacinamide or homologues of niacinamide or mixtures thereof is preferably chosen from one or more of niacin, niacinamide, picolinamide, isonicotinamide, methyl nicotinamides, cyclo-propyl niacinamide, and cyclo-pentyl niacinamide. More preferred is one or more chosen from niacinamide, picolinamide, and isonicotinamide. Most preferred is niacinamide. The composition preferably comprises 0.01 wt. % to 5 wt. %, more preferably 0.05 wt. % to 3 wt. %, furthermore preferably 0.1 wt. % to 1 wt. % vitamin B3 compound selected from niacinamide or homologues of niacinamide or mixtures thereof, by total weight of the composition.

The composition of the invention includes pectin. Pectin is generally obtained from fruits and vegetables. As per the present invention pectin is preferably obtained from a fruit selected from one or more of pears, apples, guavas, plums, gooseberries, oranges and other citrus fruits which contains large amounts of pectin. Pectin is found in the cell walls of such fruits and comprise polygalacturonic acids esterified by methoxyl groups. The composition preferably comprises 0.01 wt. % to 10 wt. %, more preferably 0.1 wt. % to 5 wt. %, further more preferably 0.5 wt. % to 3 wt. % pectin, by total weight of the composition.

Pectin may be commercially sourced as GENU® pHresh Pectin from CP Kelco, GRINDSTED® Pectin from DuPont, and Purix™ Pectin from Bakers ville.

Without wishing to be bound by theory, the present inventors believe that the combination of the actives claimed in the present invention works due to the following reason.

The skin's acid mantle is the body's first line of defense. It inhibits the growth of harmful and undesired bacteria including odour causing microorganisms while encouraging the growth of skin-healthy microflora, thus boosting the skin's defense system. The buffering and scavenging effect of pectin possibly brings the skin into balance, naturally. Pectin could be maintaining the acidic pH (acid mantle) and inhibit the growth of malodour causing bacteria, while vitamin B3 could possibly inhibit the malodour causing bacteria by slowing down the metabolic activity of the bacteria. This in combination is believed to deliver the desired benefits claimed in the present invention.

The composition of the invention comprises a cosmetically acceptable base. The cosmetically acceptable base is so chosen that the composition may be of a leave-on type. By a leave on type is meant that the composition is applied on to the skin and left thereon till the person has his next personal wash routine e.g a bath or a shower. The cosmetically acceptable base in the case of a leave-on composition is preferably in the form of a gel, emulsion, lotion, cream, stick form, or from a rollon device or delivered from an aerosol can.

### Leave-on Compositions:

An especially preferred form of the composition in a leave-on format of the invention involves a cosmetically acceptable base which is anhydrous. Such anhydrous compositions are useful for deodorant products especially for application in the underarm region. The composition comprises a topically acceptable base which is preferably anhydrous. By an anhydrous base is meant that water content in the composition is less than 5 wt. %, preferably less than 2 wt. %, more preferably less than 1 wt. % and optimally absent from the composition. To enable this, the anhydrous base preferably comprises a silicone compound, a polyhydric alcohol or a wax.

The pH of the composition is preferably higher than 3.5 more preferably in the range of 4 to 7. The pH of the composition of the invention is measured using the following procedure:

Equal volumes of the composition and model ionic sweat (pH 6.1) are mixed, and the pH value is measured using an accurate range pH test paper.

The anhydrous composition of the invention is a deodorant composition having a cosmetically acceptable base which preferably comprises a polyhydric alcohol.

Polyhydric alcohol is also referred to in short as polyol. A polyhydric alcohol as per the present invention is a compound having two or more hydroxyl groups. Suitable class of polyhydric alcohols that may be included in the composition of the invention are monomeric polyols, polyalkylene glycols or sugars. Preferred monomeric polyols are glycol; alkylene glycol e.g., propylene glycol; glycerol; or xylitol, more preferably propylene glycol.

Suitable polyalkylene glycols are polyethylene glycol or polypropylene glycol. Sugars for inclusion in the invention could be monomeric, dimeric, trimeric or of the polymeric form. Preferred sugars include glucose, fructose, mannose, sucrose, threitol, erythritol, sorbitol, mannitol, galactitol, adonitol, dextran, or cyclodextrin. Of these the more preferred sugars are glucose, fructose, sucrose, sorbitol, mannitol, adonitol, dextran, or cyclodextrin.

Other components commonly included in conventional compositions may also be incorporated in the composition of the present invention. Such components include skin care agents such as emollients, humectants and skin barrier promoters; skin appearance modifiers such as skin lightening agents and skin smoothing agents; anti-microbial agents, in particular organic anti-microbial agents, and preservatives.

The composition of the invention can be applied cosmetically and topically to the skin, broadly speaking, by one of two methods. Some consumers prefer one method and some others, the other method. In one method, sometimes called a contact method, a composition is wiped across the surface of the skin, depositing a fraction of the composition as it passes. In the second method, sometimes called the non-contact method, the composition is sprayed from a dispenser held proximate to the skin, often in an area of about 10 to 20 cm². The spray can be developed by mechanical means of generating pressure on the contents of the dispenser, such as a pump or a squeezable sidewall or by internally generated pressure arising from a fraction of a liquefied propellant volatilising, the dispenser commonly being called an aerosol.

There are broadly speaking two classes of contact compositions, one of which is liquid and usually applied using a roll-on dispenser or possibly absorbed into or onto a wipe, and in the second of which the antiperspirant active is distributed within a carrier liquid that forms a continuous phase that has been gelled. In one variation, the carrier fluid comprises a solvent for the antiperspirant and in a second variation, the antiperspirant remains a particulate solid that is suspended in an oil, usually a blend of oils.

Many different materials have been proposed as gellant for a continuous oil phase, including waxes, small molecule gelling agents and polymers. They each have their advantages and of them, one of the most popular class of gellant comprises waxes, partly at least due to their ready availability and ease of processing, including in particular linear fatty alcohol wax gellants. A gelled antiperspirant composition is applied topically to skin by wiping it across and in contact with the skin, thereby depositing on the skin a thin film.

The nature of the film depends to a significant extent on the gellant that is employed. Although wax fatty alcohols have been employed as gellant for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

The deodorant composition according to the present invention is preferably in the form of a liquid composition having a cosmetically acceptable base. Preferably the cosmetically acceptable base is selected from the group consisting of hydrophobic carrier and carrier liquid. Alternately the cosmetically acceptable base is in the form of a emulsion. Liquid compositions that are applicable from a roll-on broadly speaking can be divided into two classes, namely those in which an antiperspirant active is suspended in a hydrophobic carrier, such as a volatile silicone and those in which the antiperspirant active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right, but its popularity waned because it stings, especially if the surface onto which the composition has been applied has been damaged or cut, such as can easily arise during shaving or other de-hairing operations.

The second class of formulations that is an alternative to alcoholic formulations comprise a dispersion of water-insoluble or very poorly water-soluble ingredients in an aqueous solution of the antiperspirant. Herein, such compositions will be called emulsions. Antiperspirant roll-on emulsions commonly comprise one or more emulsifiers to maintain a distribution of the water-soluble ingredients.

The composition of the invention may be delivered through an aerosol composition which comprises a propellant in addition to the other ingredients described hereinabove. Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred. In calculating propellant to base weight ratios, the base should be considered to comprise all the components of the composition excluding the propellant.

Propellants herein generally are one of three classes; i) low boiling point gasses liquified by compression, ii) volatile ethers and iii) compressed non-oxidising gases.

Class i) is conveniently a low boiling point material, typically boiling below −5° C., and often below −15° C., and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutane. The second class of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The third class of propellant comprises compressed non-oxidising gasses, and in particular carbon dioxide or nitrogen. Inert gases like neon are a theoretical alternative.

When the composition of the invention is delivered in a roll-on, a firm solid or a stick format, the topically or cosmetically acceptable base comprises a hydrophobic carrier or an aqueous carrier. The hydrophobic carrier in such cases may comprise a silicone compound, or a wax. When the composition comprises a propellant, it is delivered as an aerosol.

The composition of the invention preferably additionally comprises a fragrance. By a fragrance is meant a molecule or a composition comprising a group of molecules that produces a pleasant odour. The composition preferably comprises a fragrance in 0.1 to 3% by weight of the composition.

The cosmetically acceptable base could also be delivered in a water containing format e.g. a gel, cream, lotion or emulsion format. One such format is where the water containing product is thickened through use of thickeners to form a gel. In the gel form the cosmetically acceptable base preferably comprises water and thickener. An oil in water emulsion, either in lotion or in cream format, is where composition preferably comprises fatty acid. In the lotion or emulsion format, the cosmetically acceptable base comprises water and fatty acid, preferably in the form of an oil in water emulsion. Lotions generally contain 3 wt. % to 8 wt. % fatty acid. C12 to C20 fatty acids are especially preferred, furthermore preferred being C14 to C18 fatty acids. The fatty acid is preferably substantially a mixture of stearic acid and palmitic acid. A mixture of stearic acid and palmitic acid in weight ratio of 55:45 is known as hystric acid. Creams are where the compositions generally comprise 8 to 24 wt % fatty acid. Part of the fatty acid may be neutralized to form soap which may be present in 1 to 10% by weight of the composition. Such cosmetically acceptable base is usually from 10 to 99.9%, preferably from 50 to 99% by weight of the composition. The cosmetically acceptable base preferably includes water. Water is preferably included in 35 wt. % to 90 wt. %, more preferably 50 wt. % to 85 wt. %, furthermore preferably 50 wt. % to 80 wt. % by weight of the composition.

A water-in-oil emulsion may also be included as the cosmetically acceptable base. The cosmetically acceptable base in such emulsions generally comprises 5 to 50 wt. % silicone elastomer, preferably 10 wt. % to 30 wt. % by weight of the base. Silicone elastomer is preferably present in 1 wt. % to 15 wt. % by weight of the composition.

Silicone elastomers differ from linear polymers because of cross-linking. Many silicone elastomers are made from linear silicone polymers that contain reactive sites along the polymer chain. Elastomers have very different physical and chemical properties from linear polymers, and the properties of elastomers depend very much on the number of cross-links. An elastomer with a relatively small number of cross-links will be very soft and will swell significantly in the presence of a compatible solvent. As the number of cross-links increases, the hardness of the elastomer increases, and the elastomer will swell to a lesser extent in the presence of solvent. A highly suitable silicone elastomers for use in the composition of the invention is DC 9045, a dimethicone crosspolymer commercially available from Dow Corning. DC 9045 is chemically a blend of cyclopentasiloxane swelling agent and dimethicone crosspolymer (12-13%).

The swelling agent is most preferably a silicone fluid or a functional silicone fluid. The swelling agent is preferably used in an amount which is in a weight ratio of 1:10 to 10:1, more preferably 1:1 to 5:1 with respect to the reaction mixture where the silicone elastomer is prepared. Swelling agent is most preferably low molecular weight silicone oil which includes (i) low molecular weight linear and cyclic volatile methyl siloxanes, (ii) low molecular weight linear and cyclic volatile and non-volatile alkyl and aryl siloxanes, and (iii) low molecular weight linear and cyclic functional siloxanes. Most preferred, however, are low molecular weight linear and cyclic volatile methyl siloxanes (VMS). By “low molecular weight” in this paragraph is meant a compound having a molecular weight from 1000 to 9000.

Other useful silicone elastomer blends which may be used in the present invention are commercially available as (i) DC 9027 (a blend of an ultra high viscosity dimethiconol and silicone elastomer in cyclopentasiloxane) available from Dow Corning; (ii) DC 9546 (a blend of high molecular weight silicone elastomer, cyclopentasiloxane and a high molecular weight linear silicone polymer) available from Dow Corning, (iii) EL8050 (a blend of high molecular weight polyglycol-modified silicone elastomer in isododecane) available from Dow Corning and (iv) EL8051 (a blend of high molecular weight polyglycol-modified silicone elastomer in isodecyl neopentanoate) available from Dow Corning.

Such water in oil emulsion composition preferably comprises 10 wt. % to 70 wt. % %, more preferably 30 wt. % to 50 wt. % water by weight of the composition.

The composition according to the invention may also comprise other diluents. The diluents act as a dispersant or carrier for other materials present in the composition, so as to facilitate their distribution when the composition is applied to the skin. Diluents other than water can include liquid or solid emollients, solvents, humectants, thickeners, and powders.

The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include:

antioxidants, binders, biological additives, buffering agents, colorants, thickeners, polymers, astringents, fragrance, humectants, opacifying agents, conditioners, exfoliating agents, pH adjusters, preservatives, natural extracts, essential oils, skin sensates, skin soothing agents, and skin healing agents.

The composition of the present invention does not include organic acid such as citric acid or ascorbic acid preferably the level of organic acid such as citric acid or ascorbic acid is less than 3 wt. %, still preferably less than 2 wt. % and further more preferably less than 1 wt. % and most preferably 0 wt. %.

The composition of the present invention does not include potassium nitrate. Preferably the levels of the potassium nitrate is less than 3 wt. %, still preferably less than 2 wt. % and furthermore preferably less than 1 wt. % and most preferably 0 wt. %.

Another aspect of the present invention relates to a method of providing malodour reduction to skin comprising the step of applying the composition of the invention on to the desired skin surface. The method is preferably non-therapeutic.

The invention will now be illustrated with the help of the following non-limiting examples:

### Examples

Examples A-D,1,2: Effect of various actives and combination of actives on malodour as measured using malodour assay:

The actives as shown in Table-1 below were subjected to malodour assay as described below:

Malodour assay using Lead acetate method:

Preparation of bacterial culture:

S. hominis is the key bacteria responsible for thiol mediated body malodour and hence the test organism used in this study was S. hominis ATCC 27844. The test organisms was grown on TSA (Tryptic Soya Agar) plates overnight (22±2 hr). The organism was not more than 3 passages removed from the original source. The cell number was adjusted by re-suspending it in a suitable amount of physiological saline sufficient to achieve the required cell number based on optical density of the cells. The optical density (OD at 620 nm) of the cells were standardized to the cell number for the test organism using a spectrophotometer. A minimum final suspension of 1.0×10\hat{ }8 CFU/mL was achieved by adjusting the OD to 0.2 in saline for the malodour assay.

Preparation of lead acetate paper:

A 1% lead acetate solution was made in distilled water. Whatmann filter paper (cut in the size that could be placed over the wells of a well plate) was dipped in the lead acetate solution. The excess solution was drained and allowed to dry in Laminar Air Flow for 30 minutes.

Preparation of actives stocks:

Stock solutions of the actives were prepared for performing the assay. A 0.5% citrus pectin was prepared by weighing 0.05 g of pectin in 10 ml sterile distilled water under magnetic stirrer for 30 min. A 1:10 dilution of this stock was prepared in sterile distilled water to achieve a final stock of 0.05% citrus pectin to perform the assay. A 1% stock of niacinamide was prepared by weighing 0.1 g in 10 ml sterile distilled water.

Assay protocol:

The malodour assay was adapted from the method of Moore et al, 2003 and modified into a 96 well plate format. 100 μl Tryptic Soy Broth (TSB) with 0.1% L-cysteine-HCl was added into the wells of the 96-well plate. 20 μl of 0.2 OD culture of S. hominis was then added into the wells. The actives were added from the stock solutions at required concentrations and made up the volume to 200 μl with addition of sterile distilled water.

The assay was done in triplicates. The lead acetate paper was placed over the wells and the lid of the 96-well plate was closed and incubated at 37° C. incubator overnight. After the incubation time, the dark coloration of the lead acetate paper was observed. Visible blackening of lead acetate in the area over the well indicated the presence of H₂S. The intensity of the colour was recorded using a reflectometer as L*a*b* values. The difference between the initial and final L*a*b* values ΔE, was used to quantify malodour formation.

$$Δ E = \sqrt{{( {L}_{i} - {L}_{f} )}^{2} + {( {a}_{i} - {a}_{f} )}^{2} + {( {b}_{i} - {b}_{f} )}^{2}}$$

The percentage malodour was calculated from ΔE measured using the above assay for the various actives or combinations. The results are summarised in the Table-1 below:

TABLE 1

| | | (%) Malodour | |
|---|---|---|---|
| Example | Active (wt %) | generated | Std Dev |
| A | Bacterial Control | 100 | 0.01 |
| B | Citrus Pectin (0.005%) | 91 | 2.3 |
| C | Niacinamide (0.05%) | 89 | 4.5 |
| 1 | Citrus Pectin (0.005%) + | 47 | 2.3 |
| | Niacinamide (0.05%) | | |
| D | Niacinamide (0.1%) | 71 | 3.5 |
| 2 | Citrus Pectin (0.005%) + | 29 | 2.1 |
| | Niacinamide (0.1%) | | |

The data in the above table indicates that the compositions as per the invention (Examples—1 and 2) show synergistic benefit over the individual values delivered by pectin alone or niacinamide alone (Examples B to D).

TABLE 2

| Example | Active wt. % | (%) Malodour generated |
|---|---|---|
| E | Bacterial Control | 100 |
| F | Sucrose (2%) | 100 |
| G | Mannose (2%) | 98 |
| H | Fructose oligosaccharide (2%) | 95 |
| I | Galactose oligosaccharide (2%) | 94 |
| J | Galactose (2%) | 90 |

The data in the above table 2 indicates that the comparative carbohydrate (mannose, sucrose, fructose oligosaccharide, galactose oligosaccharide, galactose) give higher % malodour generation as compared to pectin.

1. A deodorant composition for reducing malodour on skin, the composition comprising:
i. pectin;
ii. a vitamin B3 compound selected from the group consisting of niacinamide, homologues of niacinamide, or a mixture thereof; and
iii. a cosmetically acceptable base.

2. The deodorant composition according to claim 1, wherein the vitamin B3 compound is chosen from one or more of niacin, niacinamide, picolinamide, isonicotinamide, methyl nicotinamide, cyclo-propyl niacinamide, and cyclo-pentyl niacinamide.

3. The deodorant composition according to claim 1, wherein the pectin is derived from one or more of a fruit chosen from pear, apple, guava, plum, gooseberry, orange, and any other citrus fruit.

4. The deodorant composition according to claim 1, wherein the composition comprises from 0.01 wt. % to 10 wt. % pectin.

5. The deodorant composition according to claim 1, wherein the composition comprises from 0.01 wt. % to 5 wt. % vitamin B3 compound.

6. The deodorant composition according to claim 1, wherein the cosmetically acceptable base is selected from the group consisting of hydrophobic carrier, carrier liquid, emulsion, or an anhydrous base.

7. The deodorant composition according to claim 6, wherein the cosmetically acceptable base is an anhydrous base.

8. The deodorant composition according to claim 1, wherein the composition is delivered through a stick, in a gel form, using a roll on, or through a spray/aerosol.

9. A method of inhibiting the growth of odour causing bacteria like S. hominis while not inhibiting the growth of skin commensal bacteria like S. epidermidis comprising the step of applying the deodorant composition according to claim 1, on to a desired skin surface.

10. The method according to claim 9, wherein the desired skin surface is the axilla.

11. The deodorant composition according to claim 1, wherein the vitamin B3 compound is chosen from one or more of niacinamide, picolinamide, and isonicotinamide.

12. The deodorant composition according to claim 1, wherein the vitamin B3 compound is niacinamide.

13. The deodorant composition according to claim 1, wherein the composition comprises from 0.05 wt. % to 1 wt. % vitamin B3 compound.

14. The deodorant composition according to claim 1, wherein the composition comprises from 0.1 wt. % to 1 wt. % vitamin B3 compound.

15. The deodorant composition according to claim 1, wherein the composition comprises from 0.1 wt. % to 5 wt. % pectin.

16. The deodorant composition according to claim 1, wherein the composition comprises from 0.5 wt. % to 3 wt. % pectin.

17. The deodorant composition according to claim 1, wherein the composition has a pH higher than 3.5.

18. The deodorant composition according to claim 1, wherein the composition has a pH from 4 to 7.

19. The deodorant composition according to claim 1, wherein the cosmetically acceptable base further comprises a polyhydric alcohol.

20. The deodorant composition according to claim 19, wherein the polyhydric alcohol is selected from the group consisting of monomeric polyols, polyalkylene glycols, sugars, or a mixture thereof.
